(19) United States
(12) Patent Application Publication
(10) Pub. No.: US 2025/0352500 A1
(43) Pub. Date: Nov. 20, 2025

(54) USE OF PHENOLIC ACIDS DERIVATIVES IN TREATING ISCHEMIC STROKE

(71) Applicant: LANZHOU UNIVERSITY, Lanzhou, Gansu (CN)

(72) Inventors: Li LIN, Lanzhou, Gansu (CN); Rui WANG, Lanzhou, Gansu (CN); Zhaoyi LUO, Lanzhou, Gansu (CN)

(21) Appl. No.: 18/866,823

(22) PCT Filed: May 6, 2023
(86) PCT No.: PCT/CN2023/092591

§ 371 Date: Nov. 18, 2024

(30) Foreign Application Priority Data
May 18, 2022 (CN) 202210599845.1

(51) Int. Cl. A61K 31/192; A61K 31/6615; A61P 9/10

(52) U.S. Cl. CPC A61K 31/192; A61K 31/6615; A61P 9/10

(57) ABSTRACT

A phenolic acids derivative can be used in treating ischemic stroke. Specifically, a compound represented by the following formula (I), or a pharmaceutically acceptable salts thereof, or an optical isomer, a hydrate, a solvate, or a prodrug thereof for can be used in preparing a pharmaceutical composition for the treating and/or relieving ischemic stroke.

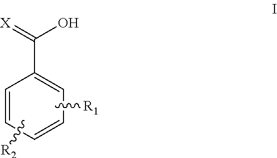

### TECHNICAL FIELD

The invention belongs to the field of life science and medicine, and specifically relates to the use of phenolic acids derivatives in treating ischemic stroke.

### BACKGROUND

Stroke is a type of disease caused by disturbance of blood circulation in the brain, leading to loss of brain function. There are two types of stroke, i.e cerebral ischemic stroke and hemorrhagic stroke, while cerebral ischemic stroke accounting for 80% of cases.

Globally, about one in six people may have a stroke, with more than 15 million new cases of stroke each year, including 5 million patients lose their lives and another 5 million patients lose their ability to take care of themselves due to permanent disability caused by stroke. A few of them can receive better treatment only.

The third national retrospective sampling survey report on the causes of death shows that cerebrovascular disease has become the leading cause of death among residents nationwide, and stroke is the disease with the highest disability rate among single diseases. For the prevention and treatment of cerebral ischemic stroke, the Chinese Stroke Prevention and Treatment Guidelines provide a series of guidance recommendations. Currently, thrombolytic therapy or intravascular thrombectomy is the most effective treatment for cerebral ischemic stroke. Even so, there are still many problems to be solved in the treatment of cerebral ischemic stroke. First, many patients miss the treatment window after onset and do not receive timely treatment. Secondly, although some patients can receive medical treatment in time, most patients still suffer from varying degrees of disability after treatment, and a small number of patients may experience exacerbation of their condition due to ischemia-reperfusion injury. Third, there is still no effective treatment for the sequelae of cerebral ischemic stroke. Therefore, it is of potential application value and important research significance to continue to study the molecular mechanisms related to cerebral ischemic stroke and to search for and develop new anti-cerebral ischemic stroke drugs.

In summary, there is still a need to develop drugs suitable for treating cerebral ischemic stroke in this field.

### SUMMARY OF THE INVENTION

The objective of the present invention is to provide a compound or a preparation containing the compound in treating ischemic stroke and its mechanism of action.

Specifically, the present invention provides a phenolic acids derivative or a pharmaceutically acceptable salts thereof, or an optical isomer, a hydrate, a solvate, or a prodrug thereof for preparing a pharmaceutical composition for the treating and/or relieving ischemic stroke. That is, the pharmaceutical composition is used in inhibiting the proliferation of astrocytes and microglia, reducing the expression level of pro-inflammatory factors consisting of TNF-α, IL-6, IL-1β, iNOS and COX2, reducing the expression level of the oxygen homeostasis regulator HIF-1α, improving and/or alleviating the inflammatory response caused by cerebral ischemic stroke and reduce the expression levels of pro-inflammatory factors (TNF-α, IL-1β, IL-6, iNOS, COX2) and HIF-1α, and inhibit the occurrence and development of inflammation, thereby achieving the effect of treating cerebral ischemic stroke and providing a new method for the treatment of cerebral ischemic stroke.

In the first aspect, the present invention provides the use of a compound represented by the following formula I, or a pharmaceutically acceptable salt, an optical isomer, a hydrate, a solvate or a prodrug thereof in the preparation of a pharmaceutical composition for treating and/or alleviating cerebral ischemic stroke:

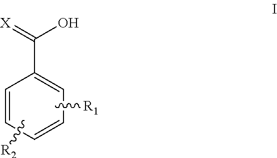

wherein X is selected from the group consisting of O and S;
R₁ and R₂ are each independently selected from the group consisting of OH, SH, NH₂, X₂—PO(OH)₂, and X₂—PS(OH)₂;
X₂ is selected from the group consisting of O and S;
[symbol] is [symbol] or [symbol] ;

Preferably, the compound of formula I has a structure selected from the group consisting of:

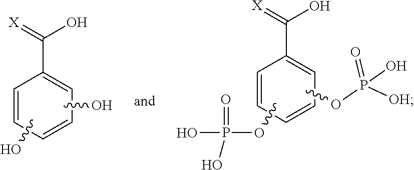

where the X is defined as above.

Preferably, the compound of formula I has a structure selected from the group consisting of:

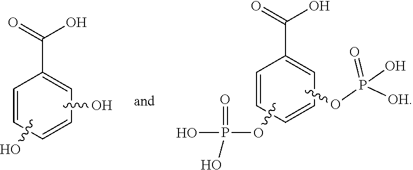

Preferably, the compound of formula I has a structure selected from the group consisting of:

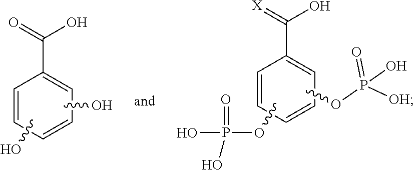

where the X is defined as above.

Preferably, the compound of formula I has a structure selected from the group consisting of:

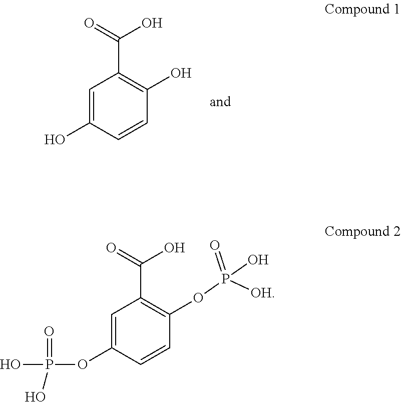 [SMILES: O=C(O)c1cc(O)ccc1O ; O=C(O)c1cc(OP(=O)(O)O)ccc1OP(=O)(O)O]

Preferably, the pharmaceutically acceptable salt is selected from the group consisting of alkali metal salts, alkaline-earth metal salts, and ammonium salts.

Preferably, the pharmaceutically acceptable salt of the compound of formula I is a pentaammonium salts of the compound of formula I.

Preferably, the pharmaceutical composition is further used in inhibiting the proliferation of astrocytes and microglia.

Preferably, the pharmaceutical composition is further used in improving and/or alleviating the inflammatory response caused by cerebral ischemic stroke.

Preferably, the pharmaceutical composition is further used in reducing the expression level of pro-inflammatory factors.

Preferably, the pro-inflammatory factor is selected from the group consisting of TNF-α, IL-1β, IL-6, iNOS, and COX2.

Preferably, the pharmaceutical composition is further used in reducing the expression level of the oxygen homeostasis regulator HIF-1α.

Preferably, the pharmaceutical composition can improve the symptoms of stroke by down-regulating the expression level of the oxygen homeostasis regulator HIF-1α.

In another aspect, the present invention provides a method for treating and/or alleviating ischemic stroke, characterized in that comprising the step of administering to a subject in need thereof a safe and effective dosage of compound 2, or a pharmaceutically acceptable salt, an optical isomer, a hydrate, a solvate or a prodrug.

It should be understood that, within the scope of the present invention, the various technical features describe above and the technical features specifically described in the appended examples can be combined with each other to form new or preferred technical solutions. Due to space limitations, all possible combinations are not explicitly set forth herein.

### BRIEF DESCRIPTION OF THE FIGURES

FIG. 1 shows the construction of MCAO model mice. (A) TTC staining results of mouse brain tissue after sham operation and cerebral ischemia-reperfusion for one day. (B) Statistical results of TTC staining results of brain tissue. (C) Laser speckle detection of cerebral blood flow. (D) Statistical results of relative cerebral blood flow (rCBF). (E) Statistical results of Zea-Longa neurobehavioral scores in mice after sham operation and ischemia-reperfusion for one day.

FIG. 2 illustrates the effect of compound 2 on the neurological function score of MCAO model mice.

FIG. 3 illustrates the effect of compound 2 on the ischemic infarction volume in MCAO mice. (A) TTC staining of the brains of mice in the Sham group, model group (MACO) and compound 2 group. (B) Statistics of the ischemic infarction volume of mice in each group.

FIG. 4 illustrates the impact of compound 2 on balance, neurological function impairment, body weight, and survival rate of model mice. (A) Statistical results of the balanced rotating rod experiment. (B) Neurological impairment on a 14-point scale. (C) Statistical results of the rate of change in weight of mouse. (D) Survival curve of mice after ischemia-reperfusion.

FIG. 5 shows the results of GFAP immunofluorescence after ischemia-reperfusion. (A) Representative images of GFAP fluorescence staining in brain tissues of the sham operation group (SHAM), model group (MCAO), compound 2 group, and compound 1 group. (Scale bar=100 μm). (B) The statistical results of the density of GFAP-positive cells in the brain tissues of the model group (MCAO), compound 2 group, and compound 1 group.

FIG. 6 shows the expression of GFAP protein in each group after ischemia-reperfusion. (A) Representative Western blot (WB) images of GFAP protein expression in brain tissues of the sham operation group (SHAM), model group (MCAO), compound 2 group, and compound 1 group. (B) Statistical results of the grayscale values of WB of GFAP protein in each group.

FIG. 7 shows the results of iba1 immunofluorescence after ischemia-reperfusion. (A) Representative images of iba1 fluorescence staining in brain tissues of sham operation group (SHAM), model group (MCAO), compound 2 group, and compound 1 group. (Scale bar=100 μm). (B) Statistical results of the density of iba1-positive cells in the brain tissues of the model group (MCAO), compound 2 group, and compound 1 group after 72 hours of ischemia-reperfusion.

FIG. 8 shows the expression of iba1 protein in each group after ischemia-reperfusion. (A) WB representative images of iba1 protein expression in brain tissues of the sham operation group (SHAM), model group (MCAO), compound 2 group, and compound 1 group. (B) Statistical results of the grayscale values of iba1 protein WB in each group.

FIG. 9 shows the results of TNF-α immunofluorescence after ischemia-reperfusion. (A) Representative images of fluorescence staining of TNF-α in brain tissues of the sham operation group (SHAM), model group (MCAO), compound 2 group, and compound 1 group. (Scale bar=100 μm). (B) Statistical results of the density of TNF-α-positive cells in the brain tissues of the model group (MCAO), compound 2 group, and compound 1 group.

FIG. 10 shows the immunofluorescence results of IL-1β after ischemia-reperfusion. (A) Representative images of fluorescence staining of IL-1β in brain tissues of the sham operation group (SHAM), model group (MCAO), compound 2 group, and compound 1 group. (Scale bar=100 μm). (B) Statistical results of the density of IL-1β-positive cells in the brain tissues of the model group (MCAO), compound 2 group, and compound 1 group.

FIG. 11 shows the immunofluorescence results of IL-6 after ischemia-reperfusion. (A) Representative images of fluorescence staining of IL-6 in brain tissues of the sham operation group (SHAM), model group (MCAO), compound 2 group, and compound 1 group. (Scale bar=100 μm). (B) The statistical results of the density of IL-6-positive cells in the brain tissues of the model group (MCAO), compound 2 group, and compound 1 group.

FIG. 12 shows the results of iNOS immunofluorescence after ischemia-reperfusion. (A) Representative images of fluorescence staining of iNOS in brain tissues of the sham operation group (SHAM), model group (MCAO), compound 2 group, and compound 1 group. (Scale bar=100 μm). (B) The statistical results of the density of iNOS-positive cells in the brain tissues of the model group (MCAO), compound 2 group, and compound 1 group.

FIG. 13 shows the results of COX2 immunofluorescence after ischemia-reperfusion. (A) Representative images of COX2 fluorescence staining in brain tissues of the sham operation group (SHAM), model group (MCAO), compound 2 group, and compound 1 group. (Scale bar=100 μm). (B) Statistical results of the density of COX2-positive cells in the brain tissues of the model group (MCAO), compound 2 group, and compound 1 group.

FIG. 14 illustrates the mRNA variation of several pro-inflammatory factors in each group after ischemia-reperfusion. Specifically, different mRNA expression levels and statistical results of TNF-α (A), IL-1β (B), IL-6 (C), iNOS (D), and COX2 (E) in the brain tissues of the sham operation group (SHAM), model group (MCAO), compound 2 group, and compound 1 group are shown.

FIG. 15 shows the expression of TNF-α, IL-1β, and IL-6 in brain tissue after ischemia-reperfusion as measured by enzyme-linked immunosorbent assay. The expression levels of TNF-α (A), IL-1β (B), and IL-6 (C) in the brain homogenate of the sham operation group (SHAM), model group (MCAO), compound 2 group, and compound 1 group, along with the statistical results.

FIG. 16 shows the expression of TNF-α protein in each group after ischemia-reperfusion. (A) Representative images of WB for TNF-α protein expression in brain tissues of the sham operation group (SHAM), model group (MCAO), compound 2 group, and compound 1 group. (B) Statistical results of the grayscale values of TNF-α protein WB in each group.

FIG. 17 shows the results of HIF-1α immunofluorescence after ischemia-reperfusion. (A) Representative images of HIF-1α fluorescence staining in brain tissues of the sham operation group (SHAM), model group (MCAO), compound 2 group, and compound 1 group. (Scale bar=100 μm). (B) Statistical results of the density of HIF-1α-positive cells in the brain tissues of the model group (MCAO), compound 2 group, and compound 1 group.

FIG. 18 shows the mRNA expression of HIF-1α after ischemia-reperfusion. The mRNA statistical results of HIF-1α in brain tissues of the sham operation group (SHAM), model group (MCAO), compound 2 group, and compound 1 group are shown.

FIG. 19 shows the expression of HIF-1α protein in each group after ischemia-reperfusion. (A) WB representative images of HIF-1α protein expression in brain tissues of the sham operation group (SHAM), model group (MCAO), compound 2 group, and compound 1 group. (B) The statistical results of the grayscale values of HIF-1α protein WB in each group.

### DETAILED DESCRIPTION

After extensive and further study, the inventor unexpectedly discovered for the first time that a compound with the structure represented by formula I, or a pharmaceutically acceptable salt, or a solvate, or a prodrug thereof, is an active ingredient that can effectively in treating and/or alleviating ischemic stroke. Experiment results have shown that compounds of formula I can inhibit the proliferation of astrocytes and microglia, reduce the expression levels of pro-inflammatory factors (TNF-α, IL-1β, IL-6, iNOS, COX2) and HIF-1α. Treating and/or alleviating ischemic stroke effect is achieved by inhibiting the occurrence and development of inflammation. The inventor completed the invention on this basis.

### Active Ingredients for Treating Cerebral Ischemic Stroke

The present invention provides the use of an active ingredient that can treat and/or alleviate the ischemic stroke. The active ingredient is a compound represented by the following formula I, or a pharmaceutically acceptable salt, an optical isomer, a hydrate, a solvate or a prodrug thereof:

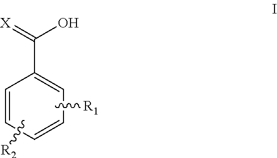

wherein the X is selected from the group consisting of O or S;
R₁ and R₂ are each independently selected from the group consisting of OH, SH, NH₂, X₂—PO(OH)₂, and X₂—PS(OH)₂.
X₂ is selected from the group consisting of O or S.
[symbol] is [symbol] for [symbol] .

Specifically, the preferred compound of formula I has a structure selected from the group consisting of:

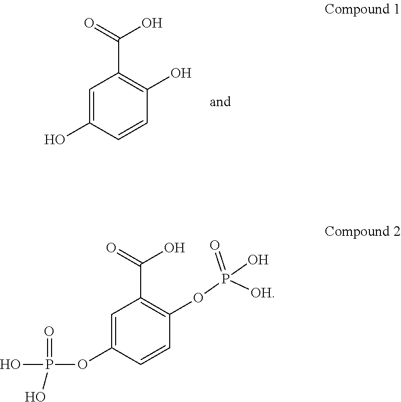 [SMILES: O=C(O)c1cc(O)ccc1O ; O=C(O)c1cc(OP(=O)(O)O)ccc1OP(=O)(O)O]

The example experiments in the present invention showed that the active ingredient can inhibit the proliferation of astrocytes and microglia, reduce the expression levels of pro-inflammatory factors (TNF-α, IL-1β, IL-6, iNOS, COX2) and HIF-1α, and achieve the effect of treating cerebral ischemic stroke by inhibiting the occurrence and development of inflammation.

As used herein, the terms “active ingredient”, “active compound of the invention”, and “active ingredient of the invention” are used interchangeably and refer to the compounds of formula I of the invention and their structural analogs.

It should be understood that the active ingredient of the invention includes the compound of formula I of the invention, or its pharmaceutically acceptable salts, enantiomer, diastereomer or racemate, or its prodrug. It should be understood that the active ingredient of the invention also includes various crystal forms, amorphous compounds, and deuterated compounds of the compound of formula I of the invention.

The “pharmaceutically acceptable salts” is the sodium salts, potassium salts, calcium salts, aluminum salts or ammonium salts formed by the compound of formula (I) and an inorganic base. or the methylamine salts, ethylamine salts or ethanolamine salts formed by the compound of formula (I) and organic base. or the corresponding inorganic acid salts formed by the compound of formula (I) with lysine, arginine, and ornithine after forming an ester, and then with hydrochloric acid, hydrobromic acid, hydrofluoric acid, sulfuric acid, nitric acid, or phosphoric acid, or the corresponding organic acid salts formed with formic acid, acetic acid, picric acid, methanesulfonic acid, or ethanesulfonic acid. In the invention, a preferred class of pharmaceutically acceptable salts is the ammonium salts, more preferably class is the pentaammonium salts.

### Pharmaceutical Compositions and Applications

The invention also provides the use of a compound of formula I, or a pharmaceutically acceptable salt, an enantiomer, a diastereomer, or a racemate thereof, and a mixture of one or more of the prodrugs thereof as an active ingredient in the preparation of a medicament for treating and/or alleviating cerebral ischemic stroke and other related diseases.

The pharmaceutical composition provided by the invention preferably contains an active ingredient in a weight ratio of 0.001-99 wt %, preferably a compound of formula I as the active ingredient accounting for 0.1 wt % to 90 wt % of the total weight, with the remaining portion being a pharmaceutically acceptable carrier, diluent, solution, or salts solution.

One or more pharmaceutically acceptable carriers can be added to the drug of the invention when it is needed. The carrier includes diluents, excipients, fillers, binders, wetting agents, disintegrants, absorption enhancers, surfactants, adsorption carriers, lubricants. Those are commonly used in the pharmaceutical field.

The compounds and pharmaceutical compositions provided by the invention can be in various forms, such as tablets, capsules, powders, syrups, solutions, suspensions, aerosols, etc., and can be present in suitable solid or liquid carriers or diluents and suitable sterile devices for injection or infusion.

Various dosage forms of the pharmaceutical composition of the invention can be prepared according to conventional preparation methods in the field of pharmacy. The unit dose of the formulation formula usually contains 0.05-400 mg of the compound of formula (I), preferably 1 mg-500 mg of the compound of formula (I).

The compounds and pharmaceutical compositions of the invention can be used clinically in mammals, including humans and animals, and can be administered through the oral, nasal, dermal, pulmonary, or gastrointestinal routes, most preferably as injectable preparations (such as infusion agents). Most preferably, the daily dose is 0.01-400 mg/kg weight, taken in one dose, or 0.01-200 mg/kg weight, taken in divided doses. No matter what kind of the method of administration, the optimal dose for individual should be determined based on the specific treatment. Usually, it is recommended to start with a small dose and gradually increase the dose until the most suitable dose is found.

The drug or inhibitor of the invention can be administered in various ways, such as injection, spraying, nasal drip, eye drip, infiltration, absorption, physical or chemically mediated methods to introduce the drug into the body such as muscle, intradermal, subcutaneous, intravenous, mucosal tissues, or mixed or encapsulated with other substances and introduced into the body.

### The Advantages of the Invention Include

(1) The compound of formula I of the invention can significantly relieve the cerebral infarction caused by ischemia-reperfusion, significantly improve the motor coordination and balance of mice with ischemia-reperfusion, significantly improve their neurological function damage, and also significantly inhibit the weight loss of mice and reduce their mortality.
(2) The experiments of the invention show that the compound of formula I can inhibit the proliferation of astrocytes and microglia, reduce the expression levels of pro-inflammatory factors (TNF-α, IL-1β, IL-6, iNOS, COX2) and HIF-1α, and achieve the effect of treating cerebral ischemic stroke by inhibiting the occurrence and development of inflammation.
(3) The compounds of the invention have low toxicity and side effects and good pharmaceutical properties.

The invention will be further described in combination with specific embodiments. It should be understood that these embodiments are only used to illustrate the invention and are not intended to limit the scope of the invention. The experimental methods in the following examples that do not specify specific conditions are usually conducted under conventional conditions or under conditions recommended by the manufacturer. Unless otherwise stated, percentages and parts are calculated by weight.

### Experimental Animal

The experiment was conducted on male C57BL/6 mice for research and brain tissue extraction. All animals used in this experiment were obtained from the Lanzhou Veterinary Research Institute of the Chinese Academy of Agricultural Sciences. Animals were raised in standard conditions: room temperature (20±2° C.), with 12 hours of alternating day and night. The experimental animals were supplied with water and standard feed, and were allowed to adapt for 3 days before the experiment. All animal experiments were conducted from 8 a.m. to 6 p.m. The animal experiments involved in this study were conducted in strict accordance with the ethical guidelines for experimental animals at Lanzhou University (License No.: JCYXY Gan 2021-0126).

Compound 1 and Compound 2 are synthesized as described by Huiyun Liu: Design, synthesis and activity evaluation of a new blood oxygen regulator [D] Lanzhou University, 2019.

### I. Construction of Cerebral Ischemic Stroke Model and Evaluation of the Efficacy of Compound 2

### Example 1: Establishment and Verification of Middle Cerebral Artery Occlusion (MCAO) Model

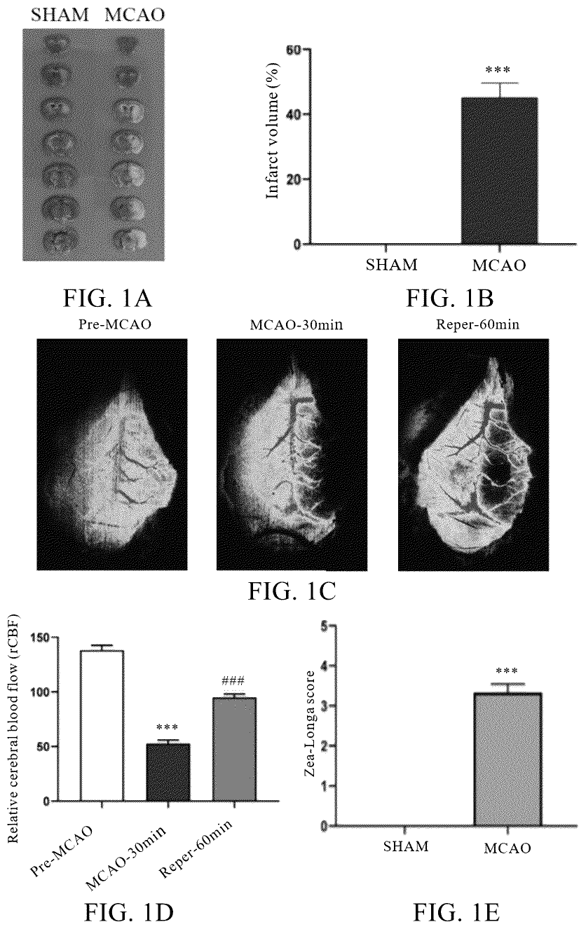

This experiment used the method of Longa et al. to construct a mouse MCAO model, which well simulates the occurrence and development of cerebral ischemic stroke disease and the treatment process in the acute phase, providing stable application value for the study of the prevention and treatment mechanism of cerebral ischemic stroke. To evaluate whether the modeling was successful, the infarct volume of the brain region was measured on the first day after surgery in mice. The experimental results are shown in FIG. 1A and FIG. 1B. The results of 2,3,5-triphenyte-trazolium chloride (TTC) staining showed that there was an infarct in the brain region one day after surgery, with an infarct volume of 45.14±4.48% (P<0.001). The blood flow at different times was measured with a laser speckle flowmeter before ischemia, during ischemia, and after reperfusion. The experimental results are shown in FIG. 1C and FIG. 1D. After 30 minutes of middle cerebral artery occlusion, the blood flow index decreased compared with before ischemia (138.3±5 vs 52.67±2.3, P<0.001). After 1 hour, the thread was pulled out and reperfusion was performed, and blood flow resumed. Compared with the ischemic period, the blood flow index increased significantly (52.67±2.3 vs 95±5, P<0.001). At the same time, the Zea-Longa scoring screening method was used to score the behavior of mice 24 hours after modeling. The experimental results are shown in FIG. 1E. On the first day after surgery, the mice showed significant stroke symptoms, with a pronounced tilt in their body when crawling, which was significantly different from the sham operation (P<0.001). Based on the TTC staining results, blood flow conditions, and mouse behavior, the MCAO model established in this study was successful. n=6, data are expressed as mean±SEM, analyzed with one-way ANOVA, and subjected to Tukeys HSD test. *P<0.05, **P<0.01, ***P<0.001, * indicates comparison with SHAM (FIG. 1B and FIG. 1E), and * indicates comparison with Pre-MCAO (FIG. 1C and FIG. 1D). ^{#}P<0.05, ^{##}P<0.01, ^{###}P<0.001, * indicates comparison with MCAO-30 min.

### Example 2: The Neurobehavioral Effects of Compound 2 on MCAO Model Mice

Male C57/BL mice weighing 23±1 g were randomly divided into sham operation group (SHAM), model group (MCAO) and drug administration groups (50 mg/kg, 100 mg/kg, 200 mg/kg), with 6 mice in each group. Mice in the SHAM group underwent vascular isolation, but no actinomycin was administered, and no treatment was given before or after surgery. The model group mice were constructed with ischemic MCAO models, and no treatment was given before and after surgery. The drug administration group was treated with compound 2 on the basis of the model group. Mice with a neurological score of 0 after reperfusion and those that died before 72 hours post-surgery were excluded. The drugs were administered through the tail vein at 0, 1, and 2 days after reperfusion. The SHAM group and MCAO group were given equal volumes of saline.

The neurological function score reflects the neurological injury status of MCAO model mice. After 24 hours of modeling, the neurological function score was performed (refer to the Zea-Longa scoring screening method). Mice with scores of 1-3 were successfully modeled, and those with scores of 0 and 4 were excluded. The scoring criteria are shown in the following table:

Zea-Longa scoring screening method

| Behavioral testing | score |
|---|---|
| When the mouse's tail is raised and suspended, its two front | 0 |
| limbs stretch straight towards the ground | |
| When the mouse tail is lifted and suspended in the air, the | 1 |
| contralateral forelimb is flexed, the shoulder is adduction, and | |
| the forearm is extended | |
| The phenomenon of turning around the opposite side of the | 2 |
| lesion | |
| Tilt to the opposite side of the surgery, unable to walk | 3 |
| No autonomic activity with conscious disturbance | 4 |

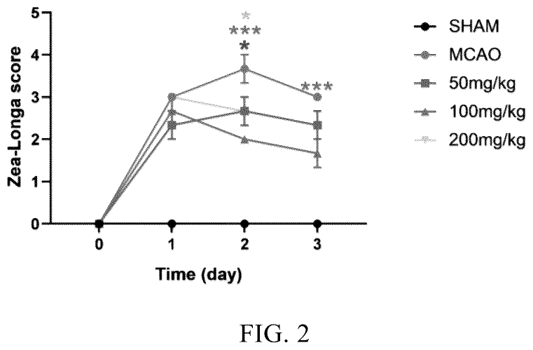

In this experiment, the Zea-Longa scoring method was used to score the sham hand group (SHAM), model group (MCAO), compound 2 (50 mg/kg) group, compound 2 (100 mg/kg) group, and compound 2 (200 mg/kg) group. The main scoring time points were before surgery, day 1, day 2, and day 3. The experimental results are shown in FIG. 2. The score of the sham operation group was 0, indicating that the mice did not experience neurological damage. Compared with the sham operation group, the MCAO group and the compound 2 group showed a significant circling phenomenon towards one side at 24 hours of reperfusion, indicating successful model construction. Compared with the model group, there was no significant difference in the compound 2 group at 24 hours of reperfusion, but after 48 hours, the scores of the three concentrations of compound 2 group decreased significantly, with the compound 2 (100 mg/kg) group showing the most significant decrease in score (P<0.001). After 72 hours of reperfusion, the neurological score at the drug concentration of compound 2 (100 mg/kg) was significantly decreased compared with the model group (P<0.001). n=6, data are mean±SEM, analyzed with two-way ANOVA and Tukeys HSD test. *P<0.05, **P<0.01, ***P<0.001, * compared with the model group (MCAO).

The results showed that compound 2 could effectively improve the neurological function deficit of model mice, improve the coordination and balance ability of mice after ischemia-reperfusion, and may have a good protective effect on repairing the damage after cerebral ischemia-reperfusion.

### Example 3: The Effect of Compound 2 on the Cerebral Infarction Volume in MCAO Model Mice

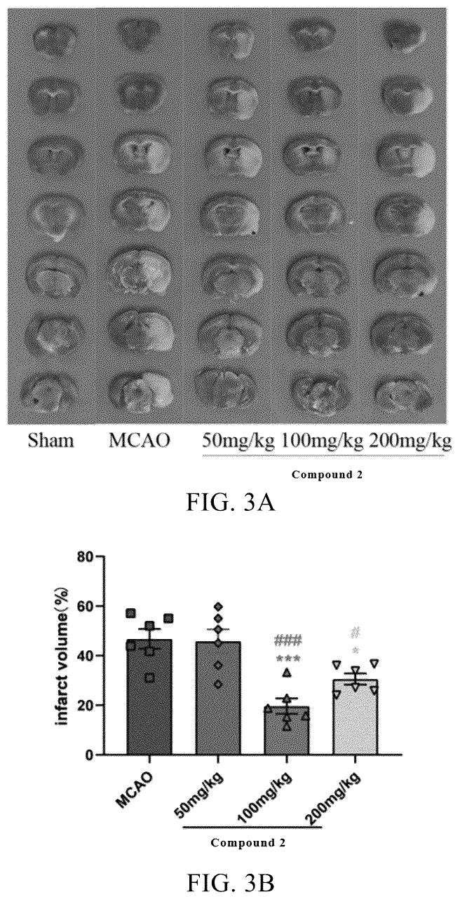

The cerebral infarction volumes of the sham-hand group (SHAM), model group (MCAO), compound 2 (50 mg/kg) group, compound 2 (100 mg/kg) group, and compound 2 (200 mg/kg) group in Example 2 were detected by TTC staining. The results are shown in FIG. 3. The brain tissue of the model group mice showed obvious ischemic infarction. The cerebral infarction volumes of mice in the different concentration groups were different. Compared with the model group, the infarction volume of model mice treated with compound 2 (100 mg/kg) was significantly reduced (27.17±5.18%, P<0.001), and the infarction volume of mice in the compound 2 (200 mg/kg) group was also significantly reduced (16.19±3.56%, P<0.05). Compared with the administration concentration of 50 mg/kg of compound 2, the cerebral infarction volume of model mice administered with a concentration of 100 mg/kg was significantly smaller (26.22±4.68%, P<0.05). n=6, data are presented as mean±SEM, analyzed with one-way ANOVA and Tukey's HSD test. *P<0.05, **P<0.01, ***P<0.001, indicating comparison with the model group (MCAO). ^{#}P<0.05, ^{##}P<0.01, and ^{###}P<0.001 indicate a comparison with compound 2 at 50 mg/kg.

### II. Mechanism Study on the Compound of the Invention in Treating Cerebral Ischemic Stroke Model

The invention takes compound 1 and compound 2 as examples to study the mechanism of the compounds of the invention in treating cerebral ischemic stroke.

Grouping and administration method: Male C57/BL mice weighing 23±1 g were randomly divided into sham operation group (SHAM), model group (MCAO), compound 2 group (100 mg/kg), and compound 1 group (50 mg/kg), with 10 mice in each group. Mice in the SHAM group underwent vascular isolation, but no actinomycin was administered, and no treatment was given before or after surgery. The model group mice were constructed with MCAO models and were not treated before and after surgery. The treatment group was treated with compound 2 or compound 1 on the basis of the model group. Mice with a neurological score of 0 after reperfusion and those that died before 72 hours post-surgery were excluded. The administration time for animal behavior tests was seven times at 0 h, 1 day, 2 days, 3 days, 4 days, 5 days, and 6 days after reperfusion, while the administration time for other experiments was three times at 0 h, 1 day, and 2 days after reperfusion. The drug was administered through the tail vein, and the sham operation group and model group were given equal amounts of saline.

### Example 4: The Effect of the Compound of the Invention on the Behavioral Function of MCAO Model Mice

After ischemia-reperfusion, neurons are severely damaged, synaptic structures are severely damaged as well. The cascade of these injuries will eventually lead to severe behavioral defects. To study the effects of compound 2 and compound 1 on behavior after ischemia-reperfusion, the balance rod test was used to test and analyze the coordination of movement in mice after ischemia-reperfusion.

The balance rod test was used to test the motor coordination of mice. The experiment was divided into four groups: sham operation group (SHAM), model group (MCAO), compound 2 group and compound 1 group. The effects of compound 2 on the recovery of behavioral function in mice after ischemia-reperfusion were comprehensively analyzed. The rotating rod test can test the motor coordination ability of mice. The experimental procedure is set to gradually accelerate the speed of the rotating rod from 10 rpm to 40 rpm within 300 seconds. Record the time that the mouse stays on the rotating stick. The mice were trained 3 days in advance. Select mice that can stay on the rotating rod for about 300 seconds for the next experiment. The time that the mice spent on the rotating rod before the model was constructed was selected as the pre-operative test value. The trained mice were subjected to MCA occlusion for 60 minutes and then reperfusion. Mice in the SHAM group, MCAO group, Compound 2 group, and Compound 1 group underwent rotating rod testing on days 1, 3, 5, and 7 post-surgeries, respectively, and the duration of their stay on the rotating rod was recorded.

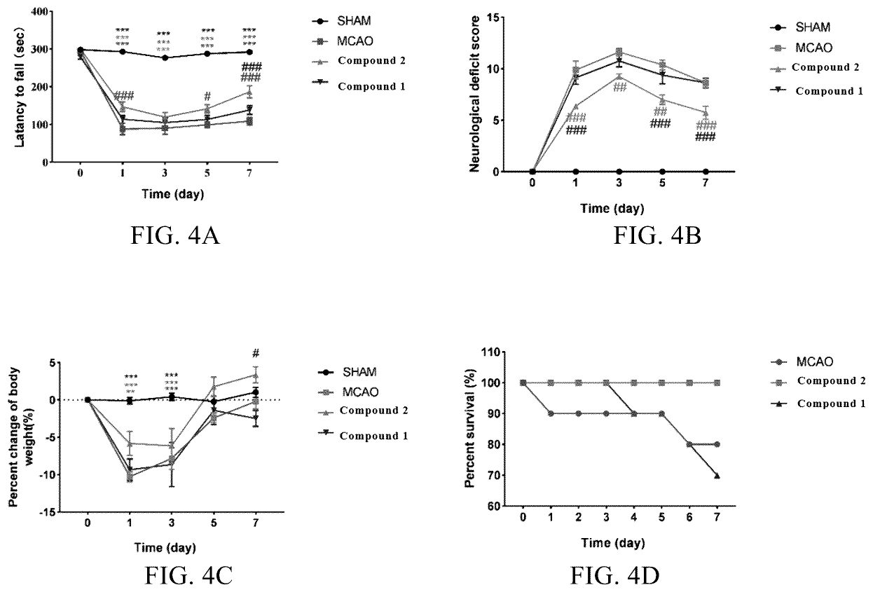

The experimental results are shown in FIG. 4A. Compared with the sham operation group, the duration of rotation in the MCAO group was significantly reduced on days 1, 3, 5, and 7 after ischemia-reperfusion (P<0.001), indicating that the behavioral damage in mice after ischemia-reperfusion was severe. After reperfusion for 1 to 3 days, the motor coordination ability continued to decline, but gradually recovered after 3 days, but the degree of recovery was extremely low. Compared with the model group (MCAO), the compound 2 group significantly improved the motor coordination ability of mice at 5 and 7 days (P<0.05), and the motor coordination of mice significantly improved at 7 days of reperfusion (P<0.001). Compared with the MCAO group, the motor coordination ability of mice in the compound 1 group also improved. Compared with the compound 1 group, the compound 2 group showed significantly improved motor coordination ability (P<0.001).

Modified Mouse Neurological Severity Score (mNSS) Table (14-point Scale)

| Behavioral sub-test | | Judgment criteria | score |
|---|---|---|---|
| (1) Test of | State of muscle | Lift the mouse by its tail: the forelimbs | 1 |
| athletic ability | hemiplegia | cannot bend | |
| | | Lift the mouse by its tail: hind legs cannot | 1 |
| | | bend | |
| | | Lift the mouse by its tail: the movement of | 1 |
| | | the head relative to the vertical axis is greater | |
| | | than 10 degrees within 30 seconds | |
| | | Place the mouse on the ground: it cannot walk | 1 |
| | | in a straight line | |
| | | Place the mouse on the ground: turn it in a | 1 |
| | | circle towards the paralyzed side | |
| | | Place the mouse on the ground: turn to the | 1 |
| | | paralyzed side | |
| | abnormal motion | motionless and fixedly gazing | 1 |
| | | quiver | 1 |
| | | Stress, epilepsy, myoclonus | 1 |
| (2) Sensory | Visual and tactile | Move the mouse to the table from the side, | 1 |
| testing | inspection | losing resistance | |
| | (Visual and | | |
| | superficial sensory | | |
| | detection) | | |
| | proprioception test | Use its claws to push against the edge of the | 1 |
| | (Deep feeling) | table to stimulate its limb muscles, causing it | |
| | | to lose its resistance | |
| | | Lack of pupillary reflex | 1 |
| | | Lack of corneal reflex | 1 |
| | | Lack of startle reflex | 1 |
| | | | 1 |

| | |
|---|---|
| Total score | 14 |

In addition, the neurological deficit score (mNSS) method shown in the table above was used to score the neurological function of each group of mice on a 14-point scale. Neurobehavioral scores were assessed in mice of the SHAM group, MCAO group, Compound 2 group, and Compound 1 group on day 1, 3, 5, and 7 post-surgery. Mice with scores of 10 to 14 indicate severe neurological damage. Mice with scores ranging from 5 to 9 indicate a moderate degree of neurological damage. A score of 1 to 4 indicates a mild degree of neurological damage. The maximum score is 14. The analysis of the neurological function injury score results is shown in FIG. 4B. Compared with the MCAO group, the neurological function of mice in the compound 2 group improved significantly from day 1 to day 7 of reperfusion. Compared with the compound 1 group, the neurological function of the compound 2 group was significantly improved.

At the same time, the weight change and survival rate of mice after ischemia-reperfusion were analyzed. The results are shown in FIG. 4C. Compared with the sham operation group, the body weight of mice after ischemia-reperfusion significantly decreased. The most severe reduction occurred at 3 days of reperfusion, and then gradually recovered, but it did not return to the weight before ischemia until 7th day. Compared with the MCAO group, compound 2 inhibited the decrease in body weight of mice after ischemia-reperfusion at 7th day of reperfusion. Compared with the compound 2 group, the body weight of mice in the compound 1 group significantly decreased at 7th day after reperfusion (P<0.05). The survival curve results are shown in FIG. 4D. Compared with the MCAO group, compound 2 significantly improved the mortality rate of mice after ischemia-reperfusion. n=10, data are presented as mean±SEM, analyzed using two-way ANOVA, and subjected to Tukeys HSD test. *P<0.05, **P<0.01, ***P<0.001, ^{#}P<0.05, ^{##}P<0.01, ^{###}P<0.001. * indicates comparison with the sham operation group (SHAM). ^{#} indicates comparison with the compound 2 group.

### Example 5: The Effect of the Compound of the Invention on Astrocytes in MCAO Model Mice

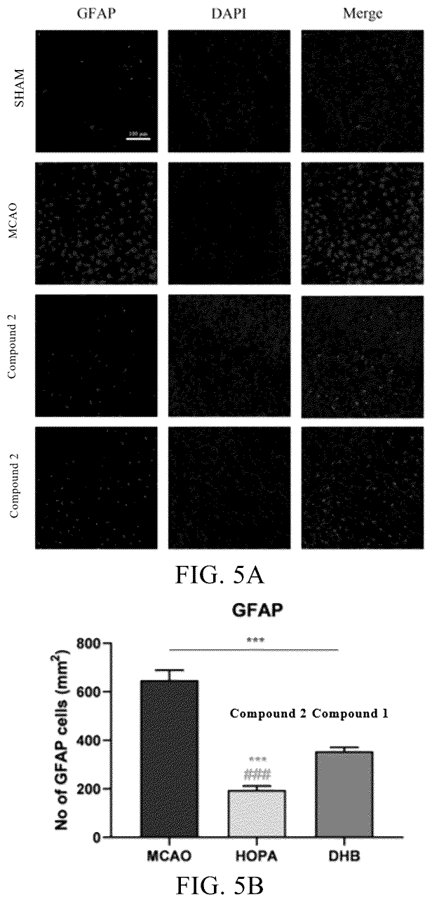

To investigate whether compound 2 has an effect on the activation and proliferation of astrocytes during ischemia-reperfusion, this study used immunofluorescence experiments to observe and detect the fluorescence expression of GFAP in brain tissues from the sham operation group, model group, compound 2 group, and compound 1 group. Subsequently, Western blot was used to detect the expression level of GFAP, a marker of astrocytes, in brain tissues. The results of fluorescence staining and quantitative analysis are shown in FIG. 5. After 72 hours of ischemia-reperfusion, the activation and proliferation of GFAP protein in astrocytes in the brain region were analyzed by immunofluorescence staining. The results of fluorescence staining are shown in FIG. 5A. A large number of GFAP-positive cells appeared in the MCAO group, while the number of GFAP-positive cells in the compound 2 and compound 1 groups significantly decreased. The statistical results are shown in FIG. 5B, where the number of GFAP-positive cells in the MCAO model group increased sharply. Compared with the MCAO group, the number of GFAP-positive cells significantly decreased after three days of administration of compound 2 (195±45 vs 481±67/mm², P<0.001). Compared with the MCAO group, the number of GFAP-positive cells also significantly decreased after three days of administration of compound 1 (327±50 vs 481±67/mm², P<0.001). Compared with the compound 1 group, the number of GFAP-positive cells in the compound 2 group decreased more significantly (195±45 vs 327±50/mm², P<0.001). n=3, data are mean±SEM, analyzed with one-way ANOVA and Tukey's HSD test. *P<0.05, **P<0.01, ***P<0.001. ^{#}P<0.05, ^{##}P<0.01, ^{###}P<0.001. * indicates comparison with the MCAO group, ^{#} indicates comparison with the compound 2 group.

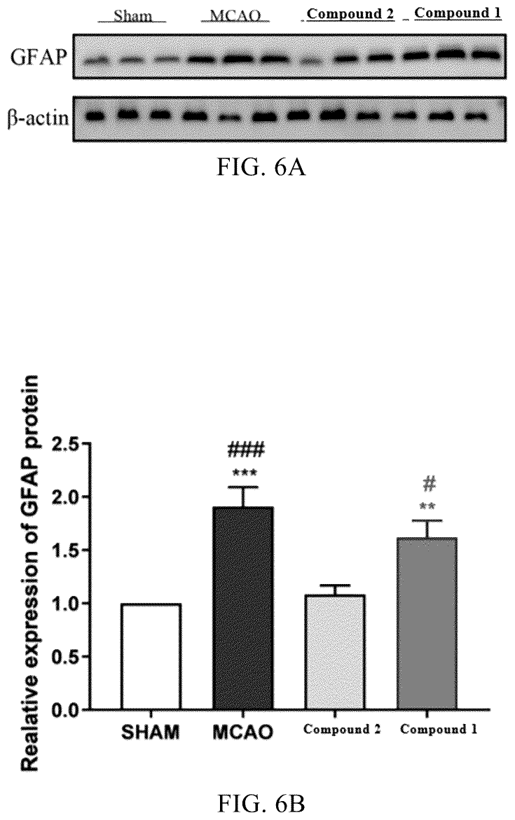

After 72 hours of ischemia-reperfusion, the protein expression level of GFAP, a marker of astrocytes in brain tissue, was detected with Western blot. The representative Western blot (WB) images of GFAP protein expression in the brain tissues of the sham operation group (SHAM), model group (MCAO), compound 2 group, and compound 1 group are shown in FIG. 6A, and the quantitative statistical results are shown in FIG. 6B. Compared with the sham operation group, the expression of GFAP protein in the brain tissue of the MCAO group increased significantly by 91±30% (P<0.001). Compared with the MCAO group, the expression of GFAP protein was significantly reduced by 43±12% (P<0.001) after three days of administration of compound 2. Compared with MCAO, the expression of GFAP protein was significantly reduced by 25±10% (P<0.01) after three days of administration of compound 1. Compared with the compound 1 group, the expression of GFAP protein in the compound 2 group was significantly reduced by 45±10% (P<0.05). In this experiment, β-actin was used as an internal reference protein. n=3, data are mean±SEM, analyzed using one-way ANOVA and Tukey's HSD test. *P<0.05, **P<0.01, ***P<0.001. ^{#}P<0.05, ^{##}P<0.01, ^{###}P<0.001. * indicates comparison with the SHAM group, # indicates comparison with the compound 2 group.

### Example 6: Effect of the Compound of the Invention on the Activation and Proliferation of Microglia in MCAO Model Mice

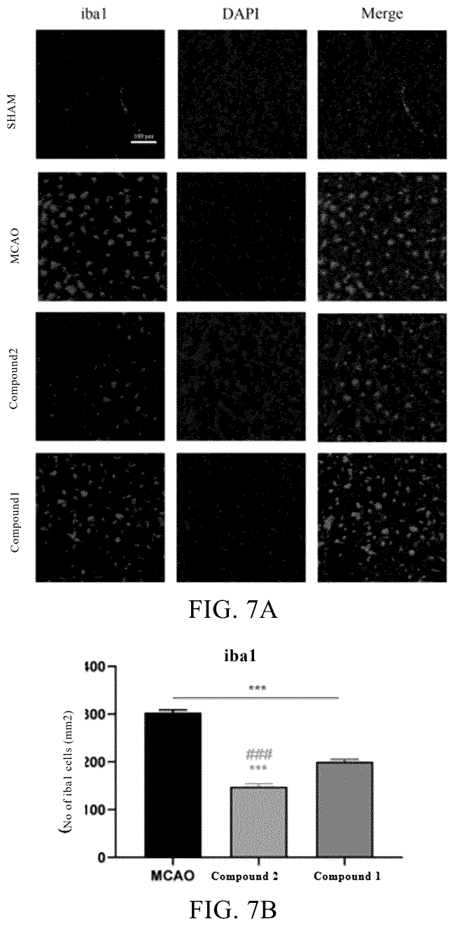

Microglia are resident phagocytic cells in the nervous system and are the primary defense system for stress and injury in central nervous cells. After cerebral ischemia-reperfusion, microglia become activated after external injury stimuli, releasing pro-inflammatory and anti-inflammatory mediators. iba1 (ionized calcium binding adapter molecule1) is specifically expressed in microglia in the central nervous system and is a calcium-binding protein of approximately 17 kDa. To investigate whether the compound has an effect on the activation and proliferation of microglia in the ischemia-reperfusion model, this study used immunofluorescence experiments to detect the fluorescence expression of iba1 in the brain tissues of the sham operation group (SHAM), model group (MCAO), compound 2 group, and compound 1 group. Then, the expression level of iba1 in microglia in brain tissue was detected by Western blot. After 72 hours of ischemia-reperfusion, the protein iba1 of microglia in the brain region was stained by immunofluorescence to analyze its changes. The fluorescence staining results are shown in FIG. 7A. The MCAO group showed a large number of iba1-positive cell signals, while compounds 2 and 1 reduced the iba1-positive cell signals. The quantitative analysis results are shown in FIG. 7B. The statistical results indicate that the expression of iba1-positive cells in the MCAO model group increased sharply. Compared with the MCAO model group, the number of iba1-positive cells significantly decreased after three days of administration of compound 2 (148±40 vs 303±23/mm², P<0.001). Compared with the MCAO model group, the number of iba1-positive cells also significantly decreased after three days of administration of compound 1 (200±12 vs 303±23/mm², P<0.001). Compared with the compound 1 group, the number of iba1-positive cells in the compound 2 group was lower (148±40 vs 200±12/mm², P<0.001). n=3, data are mean±SEM, analyzed with one-way ANOVA and Tukey's HSD test. *P<0.05, **P<0.01, ***P<0.001. ^{#}P<0.05, ^{##}P<0.01, ^{###}P<0.001. * indicates comparison with the MCAO group, ^{#} indicates comparison with the compound 2 group.

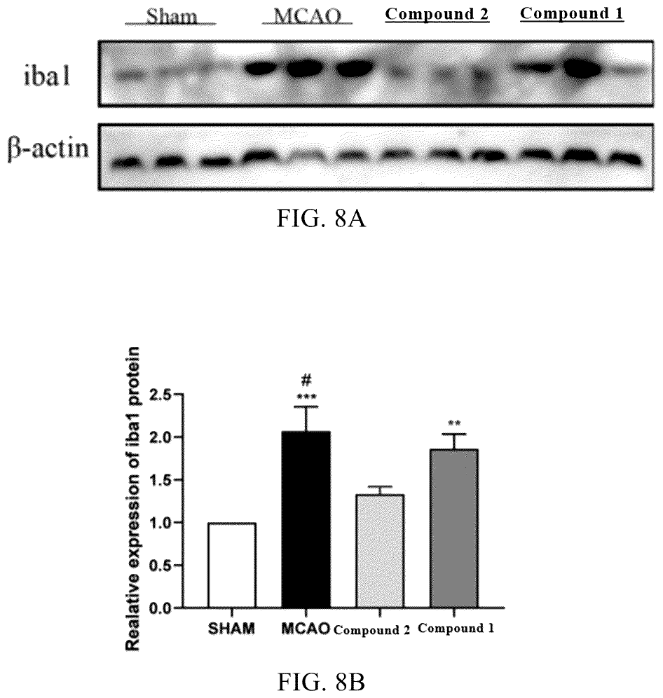

The expression level of the specific protein iba1 in microglia in brain tissues was detected by Western blot. (A) The representative WB images of iba1 protein expression in brain tissues of the sham operation group (SHAM), model group (MCAO), compound 2 group, and compound 1 group are shown in FIG. 8A, and the quantitative statistical results are shown in FIG. 8B. The results showed that compared with the sham operation group, the expression of iba1 protein in the brain tissue of the MCAO model group mice increased significantly by 110±50% (P<0.001). Compared with the MCAO group, the expression of iba1 protein was significantly reduced by 50±12% (P<0.05) after three days of administration of compound 2. Compared with the MCAO group, the expression of iba1 protein decreased three days after administration of compound 1. Compared with the compound 1 group, the expression level of iba1 protein in the compound 2 group decreased. In this experiment, β-actin was used as the internal reference protein. n=3, data are presented as mean±SEM, analyzed with one-way ANOVA, and subjected to Tukey's HSD test. *P<0.05, **P<0.01, ***P<0.001. ^{#}P<0.05, ^{##}P<0.01, ^{###}P<0.001. * indicates comparison with the SHAM group, ^{#} indicates a comparison with the Compound 2 group.

The results showed that compared with the model group, the expression levels of microglia and astrocytes in the compound 2 group and compound 1 group were significantly decreased, but compound 2 treatment could significantly inhibit the excessive activation of microglia and astrocytes in the ischemic brain region.

### Example 7: The Impact of the Compound of the Invention on the Expression Levels of Inflammatory Factors in MCAO Model Mice

The exact cause of cerebral ischemic stroke is still unclear, but research has shown that inflammatory response plays an important role in the pathogenesis of cerebral ischemic stroke. The expression changes of inflammatory factors can be used as an effective evaluation index for the therapeutic effect of ischemic brain injury. The infiltration of blood-borne leukocytes into the brain parenchyma and the activation of endogenous microglia are the causes of intense inflammatory responses after cerebral ischemia. Under ischemic conditions, the number of microglia and infiltrating immune cells in brain tissue significantly increases. Microglia promote neuroinflammation by releasing pro-inflammatory factors (TNF-α, IL-1β, and IL-6) and cytotoxic molecules (IFN-γ, prostaglandins, ROS, and NO). Microglia may cause neuronal damage through the following mechanisms: 1) secretion of pro-inflammatory factors. 2) Activating nitrogen oxides (NOX) leads to microglia proliferation and neuroinflammation. 3) expression of iNOS. 4) Endocytosis of neurons. Cyclooxygenase (COX) plays a key role in the biosynthesis of prostaglandins. COX2, as a dual-oxygenase and peroxidase, mediates the formation of prostaglandins from arachidonic acid and plays a key role in the development of inflammation. COX2 is induced and expressed after being stimulated by specific events, such as physiological stress responses to infections and inflammation, to produce prostaglandins.

To investigate the effect of compounds on the expression of inflammatory factors after ischemia-reperfusion, this study used immunofluorescence experiments to observe the expression of TNF-α, IL-1β, IL-6, iNOS, and COX2 proteins in brain tissue. RT-qPCR experiments were used to detect the mRNA gene expression of pro-inflammatory factors TNF-α, IL-1β, IL-6, iNOS, and COX2 in the sham operation group, model group, compound 2 group, and compound 1 group. The concentrations of TNF-α, IL-1β, and IL-6 in brain homogenate were detected by enzyme-linked immunosorbent assay (ELISA), and the expression level of TNF-α protein in brain tissue was detected by Western blot.

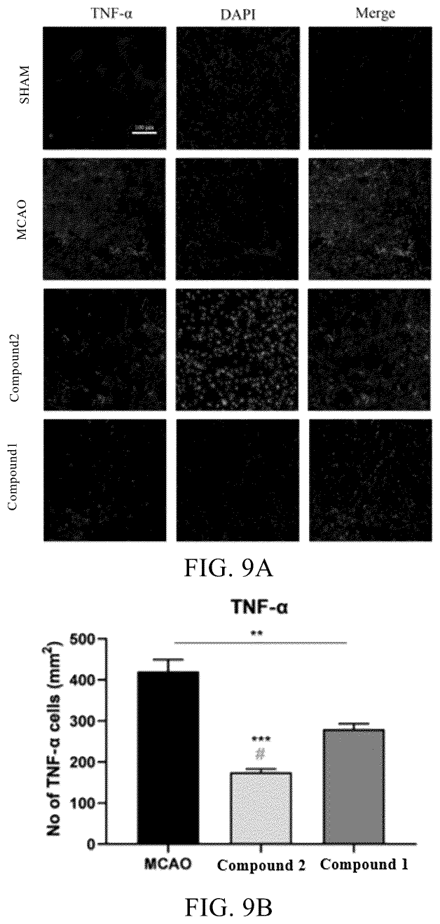

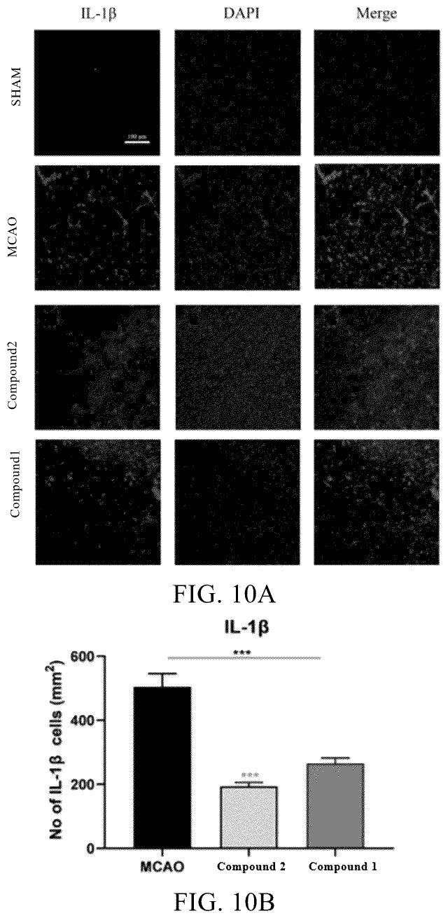

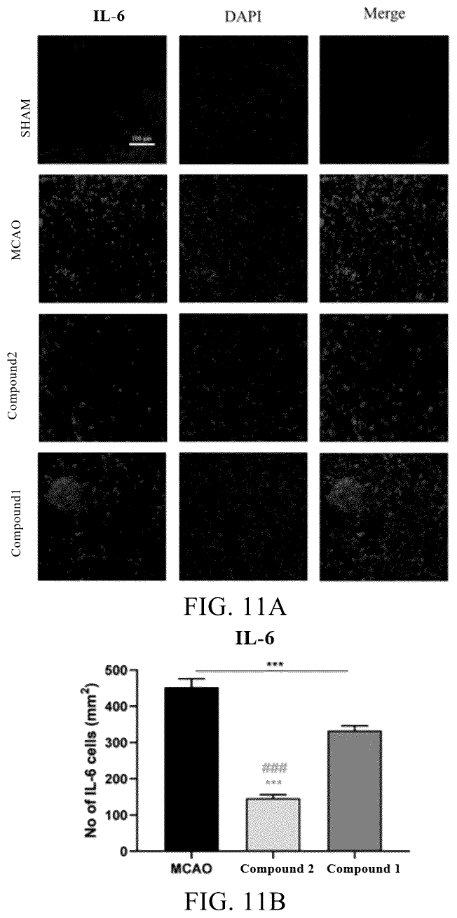

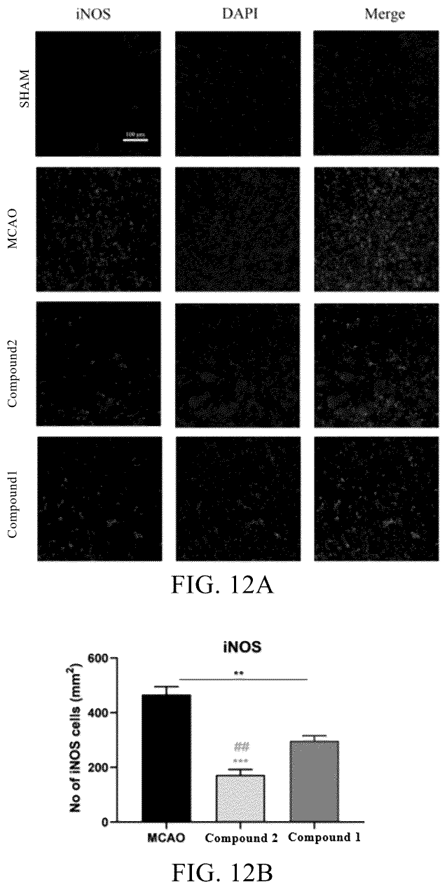

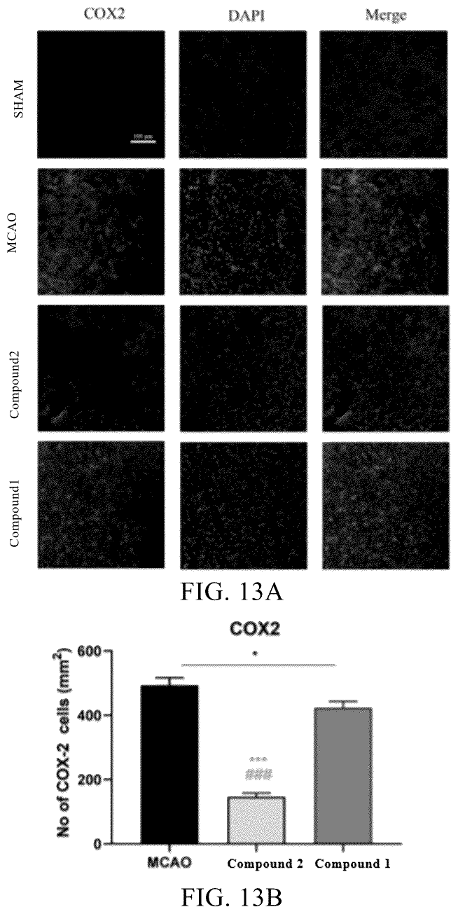

After 72 hours of ischemia-reperfusion, the expression changes of inflammatory factors such as TNF-α, IL-1β, IL-6, iNOS, and COX2 in brain regions were analyzed by immunofluorescence staining. The results are shown in FIG. 9 to FIG. 13. A large number of pro-inflammatory factor positive cell signals appeared in the MCAO group, while compound 2 and compound 1 reduced the pro-inflammatory factor positive cell signals. The statistical results showed that the number of pro-inflammatory factor-positive cells in the MCAO group increased sharply, and compared with the MCAO model group, the number of pro-inflammatory factor-positive cells significantly decreased after three days of administration of compound 2 (P<0.001). The statistical results showed that the number of iba1-positive cells in the MCAO model group increased sharply. Compared with the MCAO group, the number of pro-inflammatory factor-positive cells also significantly decreased after three days of administration of compound 1 (P<0.001). Compared with the compound 1 group, the density of pro-inflammatory factor-positive cells in the compound 2 group was significantly reduced (P<0.001). As shown in FIG. 9, after treatment with compound 2, the number of TNF-α-positive cells in MCAO model mice significantly decreased (140±35 vs 361±48/mm², P<0.001). Although the number of TNF-α-positive cells in compound 1 also decreased (244±36 vs 361±48/mm², P<0.001), the inhibitory effect was not as significant as that of compound 2 group. Other pro-inflammatory factors, including IL-1β, IL-6, iNOS, and COX2, exhibited trends similar to those of TNF-α. n=3, data are presented as mean±SEM, analyzed with one-way ANOVA, and subjected to Tukey's HSD test. *P<0.05, **P<0.01, ***P<0.001. ^{#}P<0.05, ^{##}P<0.01, ^{###}P<0.001. * indicates comparison with the MCAO group, ^{#} indicates comparison with the compound 2 group.

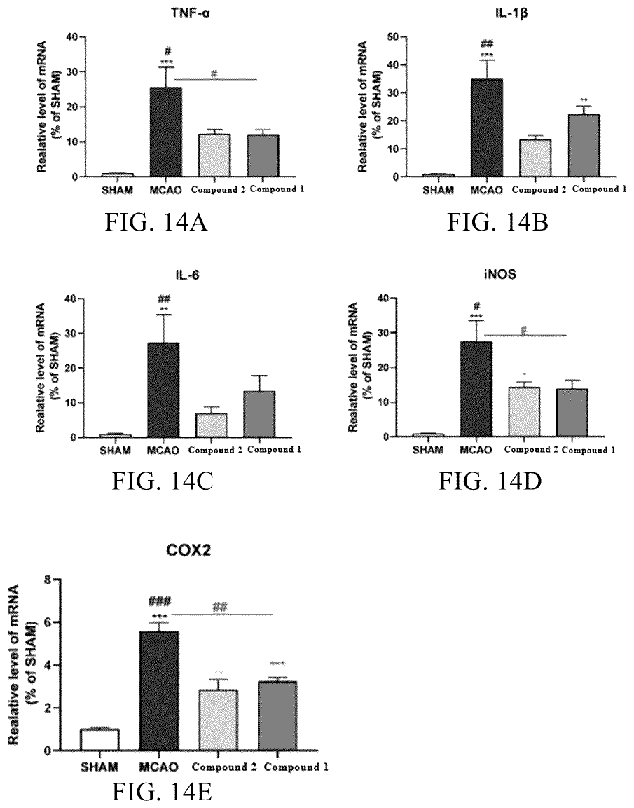

The results of the RT-qPCR experiment and quantitative analysis are shown in FIG. 14. After 72 hours of ischemia-reperfusion, the mRNA expression levels of TNF-α, IL-1β, IL-6, iNOS, and COX2 in brain tissue were detected and analyzed by RT-qPCR. The results showed that compared with the sham operation group, the expression of TNF-α (P<0.001), IL-1β (P<0.001), IL-6 (P<0.01), iNOS (P<0.001) and COX2 (P<0.001) in the MCAO group increased to varying degrees. Compared with the MCAO group, three days after administration of compound 2. The expression levels of TNF-α (P<0.05), IL-1β (P<0.01), IL-6 (P<0.01), iNOS (P<0.05), and COX2 (P<0.001) were significantly reduced. Among them, compound 2 had the most obvious inhibitory effect on the expression of IL-6, with a decrease of 51±8%. Compared with the MCAO group, the expression of TNF-α (P<0.05), iNOS (P<0.05), and COX2 (P<0.001) was significantly reduced to varying degrees after three days of administration of compound 1. n=3, data are mean±SEM, analyzed using one-way ANOVA and Tukey's HSD test. *P<0.05, **P<0.01, ***P<0.001. ^{#}P<0.05, ^{##}P<0.01, ^{###}P<0.001. * indicates comparison with the SHAM group, ^{#} indicates a comparison with the Compound 2 group.

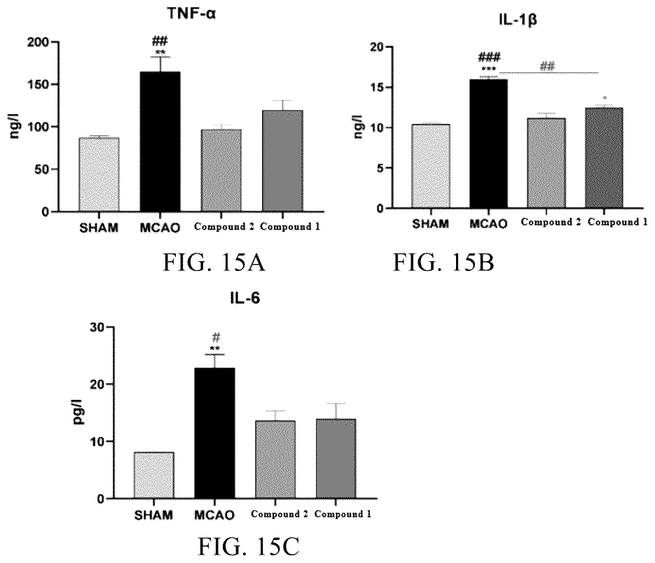

The results of the ELISA experiment and quantitative analysis are shown in FIG. 15. After 72 hours of ischemia-reperfusion, the expression levels of inflammatory factors TNF-α, IL-1β, and IL-6 in the mouse brain homogenate were detected. The results showed that compared with the sham operation group, the expression of TNF-α (P<0.01), IL-1β (P<0.001), and IL-6 (P<0.01) in the MCAO group increased to varying degrees. Compared with the MCAO group, the expression levels of TNF-α, IL-1β, and IL-6 were significantly reduced after three days of administration of compound 2, with a decrease in TNF-α expression of 40±10% (P<0.01), a decrease in IL-1β expression of 32±9% (P<0.001), and a decrease in IL-6 expression of 40±10% (P<0.05). Compared with the MCAO group, the expression levels of TNF-α, IL-1β (P<0.05), and IL-6 were reduced to varying degrees after three days of administration of compound 1. n=3, data are mean±SEM, analyzed with one-way ANOVA and Tukey's HSD test. *P<0.05, **P<0.01, ***P<0.001. ^{#}P<0.05, ^{##}P<0.01, ^{###}P<0.001. * indicates a comparison with the SHAM group, ^{#} indicates a comparison with the Compound 2 group.

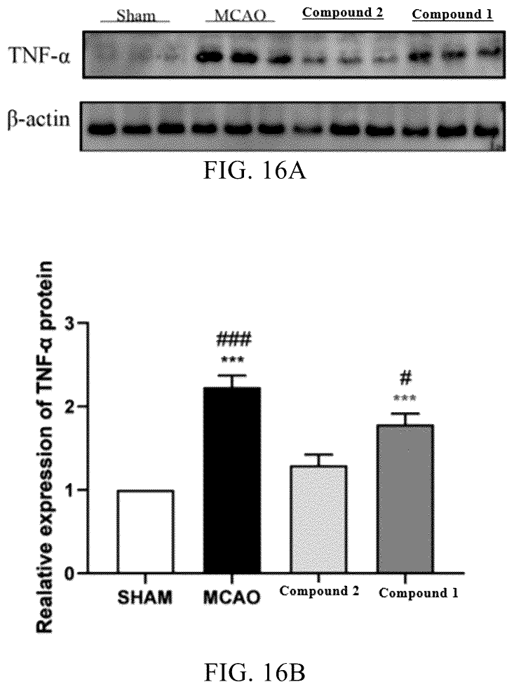

After 72 hours of ischemia-reperfusion, the expression level of TNF-α protein in brain tissue was detected by Western blot. The results of the experiment and quantitative analysis are shown in FIG. 16. Compared with the sham operation group, the protein expression of TNF-α in the brain tissue of the MCAO group increased significantly by 120±50% (P<0.001). Compared with the MCAO group, the expression of TNF-α protein was significantly reduced by 40±12% (P<0.001) after three days of administration of compound 2. Compared with the MCAO group, the expression of TNF-α protein decreased by 20±10% (P<0.001) after three days of administration of compound 1. Compared with the compound 1 group, the protein expression of TNF-α in the compound 2 group decreased by 31±20% (P<0.05). In this experiment, β-actin was used as an internal reference protein. n=3, data are presented as mean±SEM, analyzed with one-way ANOVA, and subjected to Tukey's HSD test. *P<0.05, **P<0.01, ***P<0.001. ^{#}P<0.05, ^{##}P<0.01, ^{###}P<0.001. * indicates a comparison with the SHAM group, ^{#} indicates a comparison with the compound 2 group.

In this study, the concentrations of TNF-α, IL-1β, and IL-6 in the brain homogenate of MCAO model mice were significantly higher than those in the sham operation group. After treatment with compound 2, the levels of TNF-α, IL-1β, and IL-6 in brain tissue significantly decreased, and the production of TNF-α, IL-1β, and IL-6 in the brain homogenate of the compound 1 group was also lower than that of the model group. This study suggests that compounds may inhibit the expression of inflammatory factors, reduce inflammation after ischemic cerebral infarction, and improve the damage caused by cerebral ischemia during the treatment of ischemia-reperfusion injury. Furthermore, the therapeutic effect of compound 2 is more pronounced than that of compound 1.

### Example 8: The Effect of the Compound of the Invention on Hypoxia-Inducible Factor in MCAO Model Mice

HIF-1α is a sensitive regulator of oxygen homeostasis, rapidly inducing its expression after hypoxia-ischemia. It plays a wide range of roles in the pathophysiology of stroke, including neuronal survival, neuroinflammation, angiogenesis, glucose metabolism, and blood-brain barrier regulation. It can not only produce specific sensations in the case of hypoxia, but also facilitate the maintenance of oxygen homeostasis in the body. In this study, immunofluorescence experiments were used to observe the expression level of HIF-1α in brain tissues, Western blot was used to detect the expression level of HIF-1α in brain tissues, and RT-qPCR was used to detect the gene expression of related factors in the sham operation group, model group, compound 2 group, and compound 1 group.

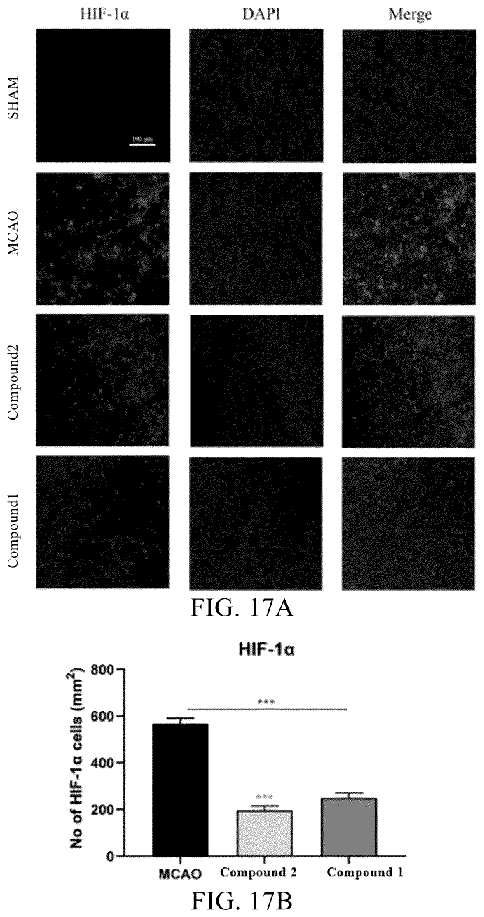

The results of fluorescence staining and quantitative analysis are shown in FIG. 17. After 72 hours of ischemia-reperfusion, the expression changes of the inflammatory factor HIF-1α in the brain region were analyzed by immunofluorescence staining. The staining results showed that there were almost no HIF-1α-positive cells in the sham operation group, while a large number of HIF-1α-positive cells appeared in the MCAO group. Compound 2 and compound 1 reduced the number of pro-inflammatory factor-positive cells. The statistical results showed that the number of HIF-1α-positive cells in the MCAO model group increased sharply compared with the sham operation group. Compared with the MCAO group, the number of HIF-1α-positive cells significantly decreased after three days of administration of compound 2 (197±33 vs 568±60/mm², P<0.001). Compared with the MCAO group, the number of HIF-1α-positive cells also significantly decreased after three days of administration of compound 1 (250±54 vs 568±60/mm², P<0.001). Compared with the compound 1 group, the number of HIF-1α-positive cells in the compound 2 group was lower (197±33 vs 250±54/mm², P<0.001). n=3, data are presented as mean±SEM, analyzed using one-way ANOVA, and subjected to Tukey's HSD test. *P<0.05, **P<0.01, ***P<0.001. ^{#}P<0.05, ^{##}P<0.01, ^{###}P<0.001. * indicates a comparison with the MCAO group, ^{#} indicates comparison with the compound 2 group.

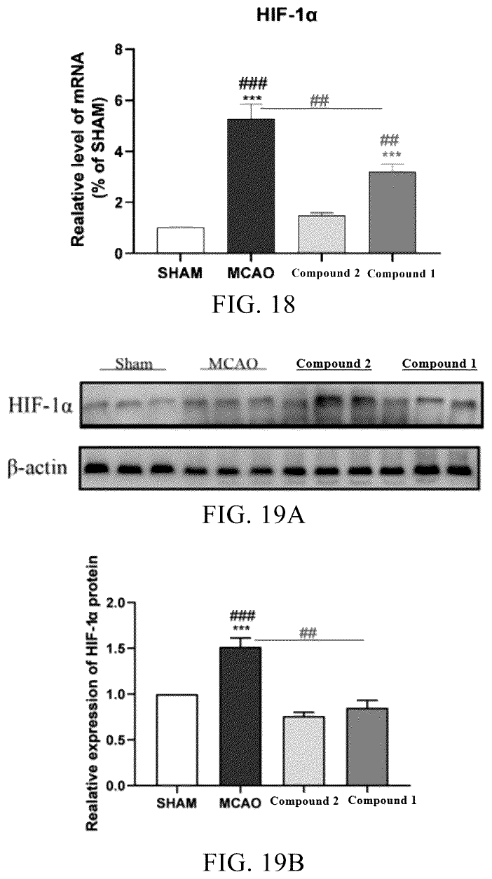

After 72 hours of ischemia-reperfusion, the mRNA expression level of HIF-1α in brain tissue was detected and analyzed by RT-qPCR. The results of RT-qPCR experiment and quantitative analysis are shown in FIG. 18. Compared with the sham operation group, the expression level of HIF-1α in the MCAO group increased significantly by 417±107% (P<0.001). Compared with the MCAO group, the expression level of HIF-1α was significantly reduced by 75±17% (P<0.001) after three days of administration of compound 2. Three days after administration of compound 1, the expression of HIF-1α also significantly decreased by 41±13% (P<0.01). Compared with the compound 1 group, the expression level of HIF-1α in the compound 2 group decreased by 53±11% (P<0.001). n=3, data are presented as mean±SEM, analyzed using one-way ANOVA, and subjected to Tukey's HSD test. *P<0.05, **P<0.01, ***P<0.001. ^{#}P<0.05, ^{##}P<0.01, ^{###}P<0.001. * indicates a comparison with the SHAM group, indicates a comparison with the Compound 2 group.

After 72 hours of ischemia-reperfusion, the expression level of HIF-1α protein in brain tissue was detected by Western blot. The experimental and quantitative analysis results are shown in FIG. 19. Compared with the sham operation group, the protein expression level of HIF-1α in the brain tissue of the MCAO model group mice increased significantly by 50±20% (P<0.001). Compared with the MCAO model group, the expression of HIF-1α protein was significantly reduced by 53±10% (P<0.001) after three days of administration of compound 2. Compared with the MCAO model group, the expression of HIF-1α protein decreased by 46±10% (P<0.01) after three days of administration of compound 1. Compared with the compound 1 group, the expression level of HIF-1α protein in the compound 2 group was significantly reduced. In this experiment, β-actin was used as the internal reference protein. n=3, data are mean±SEM, analyzed using one-way ANOVA and Tukey's HSD test. *P<0.05, **P<0.01, ***P<0.001. ^{#}P<0.05, ^{##}P<0.01, ^{###}P<0.001. * indicates a comparison with the SHAM group, ^{#} indicates a comparison with the compound 2 group.

The results showed that compared with the sham operation group, the expression of HIF-1α protein in the model group increased significantly. Compared with the model group, the expression of HIF-1α protein in the compound 2 group and compound 1 group was significantly reduced, and the reduction in the compound 2 group was more significant than that in the compound 1 group. This study confirmed through immunofluorescence experiments that compounds 2 and compound 1 can significantly reduce the expression of HIF-1α caused by ischemia-reperfusion injury. In addition, the expression of HIF-1α at the gene level was investigated by RT-qPCR experiments, and the results showed that compounds 2 and 1 significantly reduced the upregulation of HIF-1α caused by ischemia-reperfusion injury. In summary, compound 2 and compound 1 may play a role in treating ischemia-reperfusion injury by regulating the expression of HIF-1α and triggering downstream pathways.

All references mentioned in this invention are cited in this application as references, just as if each reference were cited separately as a reference. In addition, it should be understood that after reading the above description of the invention, Technicians in this field can make various modifications or modifications to the present invention, and these equivalent forms also fall within the scope of the claims attached to this application.

1. A method for treating and/or alleviating cerebral ischemic stroke, which comprises the step: administrating a pharmaceutical composition comprising compound of the following formula I, or a pharmaceutically acceptable salt, an optical isomer, a hydrate, a solvate or a prodrug thereof to a subject in need thereof; 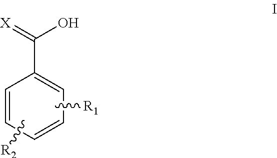
wherein X is selected from the group consisting of O and S;
R₁ and R₂ are each independently selected from the group consisting of OH, SH, NH₂, X₂—PO(OH)₂, and X₂—PS(OH)₂;
X₂ is selected from the group consisting of O and S. [symbol]
is [symbol] or [symbol] .

2. The method according to claim 1, characterized in that the compound of formula I has a structure selected from the group consisting of: 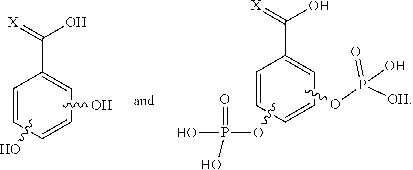
where the X is defined as above.

3. The method according to claim 1, characterized in that the compound of formula I has a structure selected from the group consisting of:

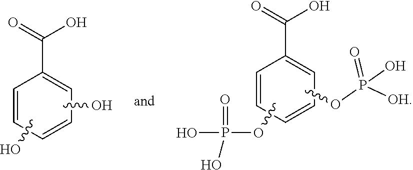

4. The method according to claim 1, characterized in that the compound of formula I has a structure selected from the group consisting of: 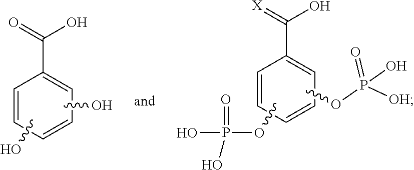
where X is defined as above.

5. The method according to claim 1, characterized in that the pharmaceutically acceptable salt is selected from the group consisting of alkali metal salts, alkaline-earth metal salts, and ammonium salts.

6. The method according to claim 1, characterized in that the pharmaceutical composition is further used in inhibiting the proliferation of astrocytes and microglia.

7. The method according to claim 1, characterized in that the pharmaceutical composition is further used in improving and/or alleviating the inflammatory response caused by cerebral ischemic stroke.

8. The method according to claim 7, characterized in that the pharmaceutical composition is further used in reducing the expression level of pro-inflammatory factors.

9. The method according to claim 1, characterized in that the pro-inflammatory factor is selected from the group consisting of TNF-α, IL-6, IL-1β, iNOS and COX2.

10. The method according to claim 1, characterized in that the pharmaceutical composition is further used in reducing the expression level of the oxygen homeostasis regulator HIF-1α.
